(19) United States
(12) Patent Application Publication
(10) Pub. No.: US 2020/0013491 A1
(43) Pub. Date: Jan. 9, 2020

(54) Interoperable Record Matching Process

(71) Applicant: ChartSpan Medical Technologies, Inc., Greenville, SC (US)

(72) Inventors: Patrick Carter, Greenville, SC (US); Brian Crow, Greenville, SC (US); Rick Lawson, Greenville, SC (US)

(73) Assignee: ChartSpan Medical Technologies, Inc., Greenville, SC (US)

(21) Appl. No.: 16/493,615

(22) PCT Filed: Mar. 9, 2018
(86) PCT No.: PCT/US18/21834

§ 371 Date: Sep. 12, 2019

Related U.S. Application Data
(60) Provisional application No. 62/470,628, filed on Mar. 13, 2017.

(51) Int. Cl. G16H 10/60; G06F 16/2455

(52) U.S. Cl. CPC G16H 10/60; G06F 16/2455

(57) ABSTRACT

An interoperable record matching process may allow for records without unique identifiers to be matched to a patient. A method may comprise receiving an electronic healthcare record comprising patient identification data and medical data; parsing the electronic healthcare record and extracting the patient identification data to produce extracted patient identification data; normalizing the extracted patient identification data to produce normalized patient identification data; retrieving a patient data file from a database wherein the patient data file comprises database patient identification data; comparing the normalized patient identification data to the database patient identification data using a string distance function, wherein the string distance function computes a string distance; comparing the distance metric to a threshold; and adding the medical data to the database and associating the medical data with the database patient identification data if the string distance meets or exceeds the threshold.

### CROSS-REFERENCE TO RELATED APPLICATIONS

This application is a non-provisional application which claims the benefit of priority to provisional application U.S. 62/470,628 filed Mar. 13, 2017.

### STATEMENT REGARDING FEDERALLY SPONSORED RESEARCH OR DEVELOPMENT

Not applicable.

### BACKGROUND OF THE INVENTION

### Field of the Invention

This invention relates to the field of data recognition and data indexing and more specifically to the field of record matching and data indexing from digital medical records.

### Background of the Invention

Digital medical records may suffer from many drawbacks including incomplete or wrong information and non-standard templates or formats. For example, a name may be misspelled in a record and consequently a computer system may not associate the record with the correct person. Non-standard templates and formats may lack identifying information, which may cause the record to not be correctly associated. Additionally, individual records generated by an organization may not be readily available to another organization due to differences in software and data formats.

Consequently, there is a need in the art for matching records with non-unique identifying information, and there is a need for an improved data record and matching solution that may recognize and interpret non-standard formats, detect errors, and correctly associate records with a person.

### BRIEF SUMMARY OF SOME OF THE PREFERRED EMBODIMENTS

These and other needs in the art are addressed in one embodiment by an interoperable record matching process that comprises a computer system capable of analyzing a record and matching the record to a person and organization. The computer system may be configured to parse a record for various identifiers, recognize the identifiers, and link the identifiers to a person or organization thereby linking the record to the person or organization. Once the identifiers are linked, data in the record may be associated with the person and organization.

An embodiment may comprise retrieving a record from a network, cloud storage, e-mail, or software. The record may then be parsed for unique identifiers such as an organization identifier or person identifier. Identifiers may be a number or other identifier that uniquely identifies the person or organization. If no identifiers are present, the record may be parsed for other non-unique identifiers such as name, address, date of birth, telephone number, email address, among others. The non-unique identifiers may be compared against a database and a string distance function, such as a Jaro-Winkler distance function, may be calculated for each non-unique identifier. An output of the string distance function may be compared against a threshold value and a non-unique identifier may be deemed to match an existing record if the output of the string distance function is greater than or equal to the threshold value. The record may then be associated with the person or organization.

The record may comprise an electronic healthcare record in a clinical data architecture format. The clinical document architecture may be in a syntactically interoperable format such as XML.

The foregoing has outlined rather broadly the features and technical advantages of the present embodiments in order that the detailed description that follows may be better understood. It should be appreciated by those skilled in the art that the conception and the specific embodiments disclosed may be readily utilized as a basis for modifying or designing other embodiments for carrying out the same purposes of the present invention.

### DETAILED DESCRIPTION OF THE PREFERRED EMBODIMENTS

Medical providers and healthcare organizations are often required by policy or regulation to keep records of access to medical histories. In some instances, it may be required that a healthcare organization attest to a government entity that the records have been kept secure and accessed only by approved entities. Some federally mandated programs such as Merit-Based Incentive Payment System (MIPS) and Alternative Payment Models (APM's) require records of access in order for a healthcare organization to be compensated for provided services. In embodiments described herein, a system may be configured to match electronic healthcare records to a patient and thereafter record any actions that are performed on a healthcare record. Actions may include viewing, recording a change, downloading, or transmitting the record. The systems described herein may allow a healthcare organization to more readily comply with federal mandates and regulations. As used herein, the term healthcare organization may refer to any organization involved in healthcare. Some examples may include, but are not limited to, hospitals, clinics, doctors, imaging centers, emergency rooms, medical records management companies, medical billing companies, medical insurance companies, and any other organizations who are involved with healthcare.

Federal mandates may require healthcare providers to allow patients access to their healthcare records and to allow patients to transfer their healthcare records between healthcare providers. In previous medical record management systems, patient records may have been paper based with little to no computerized record management. A patient may have asked for a copy of their medical record from a medical provider and the medical provider may have provided the original papers or a paper copy thereof. A new medical provider, such as a new doctor or other healthcare professional, may then have received the medical records. Medical records may comprise any kind of documents relating to a patient such as, without limitation, diagnosis records, imaging records such as x-ray images, computer aided tomography images, and nuclear magnetic resonance images, surgical records, medical history records, medication and prescription records, and other records well known in the art.

The medical provider may readily understand the content of a document in the medical record without regards to the layout or format of the document. As one of ordinary skill in the art will understand, there may be many fields in a document such as, for example, patient name, patient address, patient telephone number, patient unique identifier number, as well as other medical data content fields such as medical diagnosis, vital sign measurements, medical images, medical history, doctor notes, and others medical data well known and practiced in the art. The new medical provider may understand the content of a medical record regardless of where, for example, a patient's name is located on the document. A medical provider may readily recognize unstructured data in a document and mentally assign context and meaning to the unstructured data. In the instance of a patient's name, a medical provider should be able to recognize that a particular series of letters is the patient's name regardless of where on the paper document the name is. For example, a medical provider should recognize the patient's name if it is written at a top or bottom of the paper document. A person of ordinary skill in the art, reading a paper medical record, may be agnostic to the particular format of any fields in the medical record as they should be able to assign meaning and context to data in the fields. A paper record may be generally referred to as interoperable between healthcare providers as the content of any particular paper medical record should be recognizable to one of ordinary skill in the art.

While paper medical records may have benefits such as interoperability to those of ordinary skill in the art, there may also be drawbacks to paper medical records. Paper medical records may, for example, be difficult to transport as they require physically moving the paper medical record between locations, paper medical records may be handwritten and by extension, poorly legible which may contribute to medical errors, and paper medical records may become lost in storage. To remedy these and many other drawbacks to paper medical records, electronic medical records, also referred to as electronic healthcare records (EHR), have been developed.

Growing availability of computer resources and electronic healthcare records software has allowed healthcare providers to switch from paper medical records to electronic healthcare records. An electronic healthcare record may comprise a computer file with a file format that may be readable by electronic medical record software. The computer file may be of any format or file type. In some embodiments, the computer file may comprise data such as headers identifying file type as well as data comprising an electronic healthcare record. Data comprising the electronic health record may be identified in the computer file by tags, headers, magic numbers, or any other method well known in the art.

Although electronic healthcare records may remedy many of the drawbacks of paper medical records such as physical transport and handwritten records, electronic healthcare records may have other drawbacks that are not present in paper medical records. In particular, interoperability may be lost with the use of electronic healthcare records. While a human medical practitioner may readily recognize contents of a paper medical record and assign meaning to the content regardless of any particular format the contents are presented, electronic healthcare record software may not automatically recognize content. For example, if electronic healthcare record software is presented computer file comprising an electronic health record and the electronic healthcare record contains an incorrectly spelled patient name, the electronic healthcare record may not be associated with the patient. Consequently, the electronic healthcare record may be discarded because the record cannot be associated with any particular patient. Some medical privacy regulations, such as Health Insurance Portability and Accountability Act (HIPPA), may require destruction of electronic records that cannot be positively associated with a particular patient to prevent medical records from being accessed by anyone other than the patient and their medical care providers. In the example of a misspelled patient name in a paper medical record, a human medical provider may look for other identifying information in the paper medical record to positively identify the correct patient to associate the paper medical record with.

A healthcare provider may generate an electronic healthcare record during a patient visit. The electronic healthcare record may be generated by electronic medical record software with input from a user such as a doctor. In embodiments, the electronic medical record software may generate an electronic healthcare record in XML format. A clinical document architecture may be a particular implementation of XML used to define encoding, structure, and semantics of electronic health records. An example of a particular clinical document architecture is HL7 CDA®, published by Health Level Seven International, Inc.

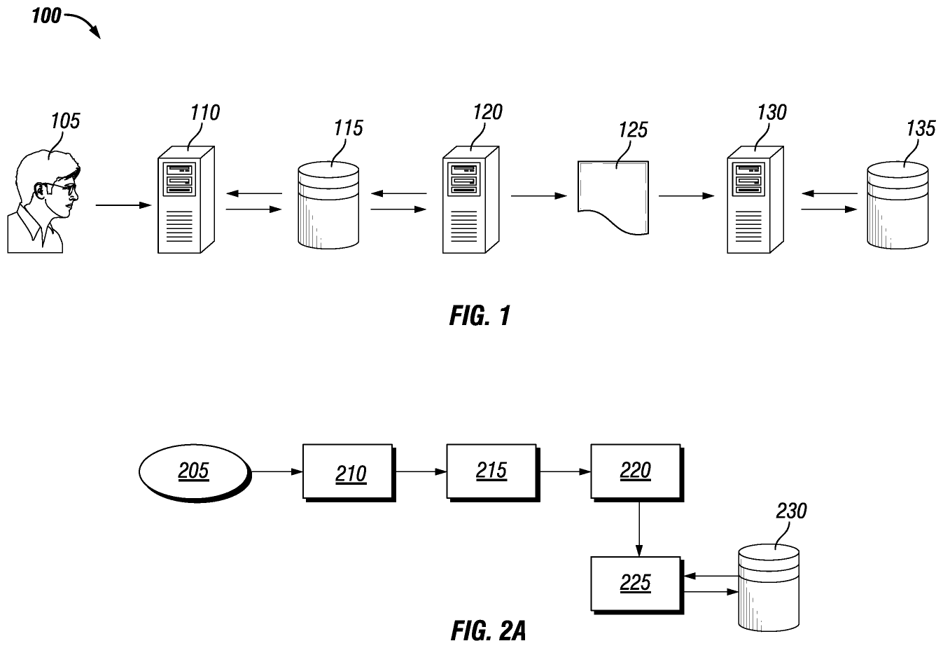

FIG. 1 illustrates an embodiment of a method 100 in accordance with the present disclosure. Method 100 may begin with healthcare provider 105. Healthcare provider 105 may be any personnel involved with a patient including, but not limited to, doctors, nurses, surgeons, medical staff members, receptionists, accountants, or any other personnel who may be involved in medical treatment of a patient. Healthcare provider 105 may use a computer 110 to generate an electronic healthcare record as previously disclosed. Electronic healthcare records may be stored in a database 115. A server 120 may interface with database 115 to provide access to electronic healthcare records stored in database 115. As previously disclosed, electronic healthcare records may need to be transferred between healthcare organizations or to a patient requesting their healthcare records. Server 120 may comprise an application programming interface (API) that allows retrieval of healthcare records stored in database 115. Electronic healthcare record 125 may be retrieved from database 115 by server 120 and sent to server 130. Server 130 may be controlled by a separate healthcare organization who requested electronic healthcare record 125. Server 130 may interface with database 135 and which may store a copy of electronic healthcare record 125.

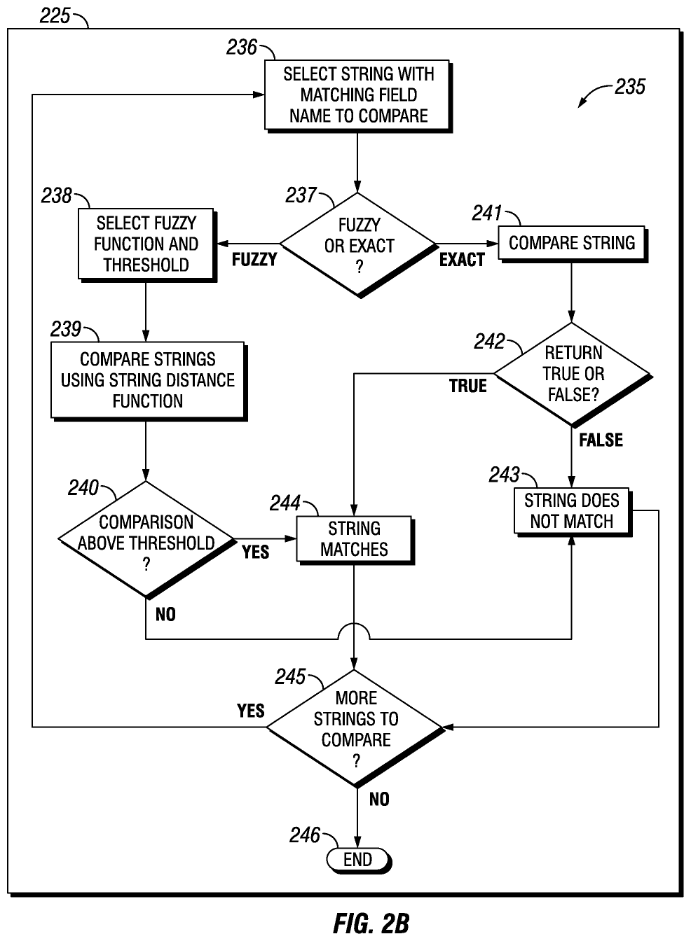

FIG. 2a illustrates an embodiment of a method 200 in accordance with the present disclosure. Method 200 may start at block 205. In block 205, an electronic healthcare record may be received by a healthcare organization as previously disclosed. In some embodiments, the electronic healthcare record is an XML based file comprising a clinical document architecture. The electronic healthcare record may be of any type and from any source. Electronic healthcare records may come from many sources such as including, but not limited to, a healthcare organization database, a Direct Trusted Protocol Network, a Blue Button+network, an application program interface (API), e-mail server in the form of an e-mail or e-mail attachment, cloud storage, electronic heath record (EHR) software, or combinations thereof. The direct trusted protocol network is a framework that allows healthcare providers and organizations the ability to share data directly in a secure manner. The Blue Button+network is a network of healthcare records that are accessible by a patient through a portal. Healthcare software may comprise an API that may be queried for records. EHR software may be used by healthcare professionals to review and edit a patient's electronic record of medical history, diagnoses, medications, radiology images, among other data. In embodiments, the electronic healthcare records may comprise patient identification data as well as medical data. Patient identification data may comprise name, address, phone number, unique patient identifiers, and other patient identification data. Medical data may comprise any other data in the electronic healthcare record such as medical diagnosis, vital sign measurements, medical images, medical history, doctor notes, and others medical data well known and practiced in the art. The electronic healthcare record may be loaded into memory of a computer where further operations may be performed on the electronic healthcare record.

In block 210, the electronic healthcare record may be parsed to extract data from the electronic healthcare record. The electronic healthcare record may be stored in memory of a computer during parsing. Parsing may, for example, extract strings of data from the electronic healthcare record such as patient name, date of birth, address, zip code, phone number, Unique Organization Identifier (UOI), a Healthcare Provider National Provider Identifier (NPI), a Unique Patient Identifier (UPI), or any other data in the health care record. The extracted strings may be stored in a memory object, such as a data structure for further processing. In an embodiment, the data is stored in an array.

In block 215, the extracted data from block 210 may be normalized. Normalization may comprise performing a series of operations on the extracted data that transforms the data into a single canonical form. Some operations may include, without limitation, removing all characters from extracted data except for letters and numbers, transforming uppercase letters into lowercase letters or vice-versa, removing white space characters, removing glyphs such as diacritic marks from letters to generate basic glyphs, and other normalization operations. One of ordinary skill in the art with the benefit of this disclosure should be able to select an appropriate normalization operation for a particular set of extracted data.

An address may be normalized according to a postal address verification standard. For example, the United States Postal Service (USPS) publishes a standard known as coding accuracy support system (CASS). CASS and other address normalization standards may correct errors in mailing addresses by filling in missing parts of addresses and formatting an address to a defined appearance. A postal address verification standard may perform several normalization operations, including, without limitation, completing a zip code based on a city and street address, normalize abbreviations such as lane to LN, and correct misspellings in street names. One of ordinary skill in the art, with the benefit of this disclosure, should be able to select and appropriate postal address verification standard and apply it to a particular set of extracted data.

A date of birth may be normalized by comparing a date of birth from the extracted data to a list of address formats, determining the format of the date of birth from the extracted data, and reordering the data of birth from the extracted data to a desired structure. For example, a birthdate may be presented as yyyyMMdd which corresponding to year, month, and day. Alternatively, without limitation, a birthdate may be presented as yyyyddMM, ddMMyyyy, ddyyyyMM, MMddyyyy, or MMyyyydd. Normalizing a birthdate may comprise comparing a string comprising a birthdate, after all characters except for number have been removed, to a list of birthday formats to determine the format of the string comprising a birthdate, and then rearranging the format to a desires structure. For example, a string may be presented as ddMMyyyy which may then be normalized to yyyyMMdd. One of ordinary skill in the art, with the benefit of this disclosure, should be able to select and appropriate method to noimalize a date of birth and apply it to a particular set of extracted data.

In block 225 the normalized data from 215 is compared to a dataset retrieved from database 230. Database 230 may comprise a healthcare organization's database of patient electronic healthcare records. Patient electronic healthcare records may comprise a list of patients and medical data associated with each patient in the patient list. A dataset retrieved from database 230 may comprise a list of patients. Comparisons may be performed by a fuzzy comparing function or an exact comparing function. A fuzzy comparing function may be a string distance function. A string distance function may be a metric that measures a distance between two strings. Some string distance functions may include, without limitation, Levenshtein distance, Jaro-Winkler distance, Damerau-Levenshtein distance, and Hamming distance. A string distance function may output a number indicating an algorithm specific indication of distance or similarity between two strings.

For example, Jaro-Winkler distance will output a number between 0 and 1. An output of 0 indicates the strings are completely dissimilar For example, comparing the strings “cat” and “dog” using Jaro-Winkler distance would return an output of 0. An output of 1 indicates the strings being compared are the same. For example, comparing the strings “cat” and “cat” using Jaro-Winkler distance would return an output of 1. In another example, Levenshtein distance may calculate the number of deletions, insertions, or substitutions required to transform a source string into a target string. Comparing the strings “book” and “back” using Levenshtein distance would output 2. Damerau-Levenshtein may extend Levenshtein distance to include transpositions of characters. Comparing the strings “John” and “Jonh” using Damerau-Levenshtein distance would output 1. In another example, a Hamming distance may calculate the number of positions at which the corresponding symbols are different. The Hamming distance output the minimum number of substitutions to change one string to another. Comparing the strings “John” and “Jonh” using Hamming distance would output 2. For any selected string distance function, the output of the function may vary depending on the exact input parameters of the string distance function and any modifications to the string distance function. One of ordinary skill in the art with the benefit of this disclosure should be able to select an appropriate string distance function and understand an output of the selected string distance function.

As previously discussed, there may be errors in an electronic healthcare record such as misspelled names, which may cause an output of a string distance functions to indicate that the compared strings do not match. A threshold or cutoff may be selected such that strings that are compared and deemed similar enough may be considered a match. In the example of a Jaro-Winkler distance which outputs between 0 and 1, a threshold of greater than 0.92 may be selected. Alternatively, a threshold of greater than 0.7, 0.8, 0.9, 0.99, or any values between may be selected. A threshold may be selected based on the specific string distance function selected to compare strings. One of ordinary skill in the art with the benefit of this disclosure should be able to select an appropriate threshold value for a particular string distance function selected.

As previously disclosed, comparisons may be also be performed by an exact comparing function. An exact comparing function may be a string distance function as previously described with a threshold set to only indicate a match when the compared strings are exactly matching.

Once the normalized data from 215 is compared to a dataset retrieved from database 230, the electronic healthcare record may be determined to match and the electronic healthcare record may be added to database 230. If the electronic healthcare record is determined to not match, the record may be discarded.

FIG. 2b illustrates an embodiment of the method 235 of block 225 in FIG. 2a. Method 235 may start with block 236 by selecting strings with a matching field name or field type from normalized data provided in block 215 of FIG. 2a and dataset retrieved from database 230 of FIG. 2a. For example, the field name may be first name. Strings from field names matching first name may be compared. The method may proceed to decision block 237 where the comparing function is selected. As previously disclosed, the comparing function may be fuzzy or exact. If the fuzzy comparison is selected, method 235 may proceed to 238 where a string distance function and threshold may be selected. In block 239 the strings may be compared using the selected string distance function. In decision block 240 the output of the selected string distance function is compared to the selected threshold. If an output of the selected string distance function is above the selected threshold, the strings may be determined to match and method 235 may proceed to block 244. If the output of the selected string distance function is below the selected threshold, the strings may be determined to not match and method 235 may proceed to block 243. If the exact comparison function is selected in decision block 237, method 235 may proceed to block 241 where the strings are compared. As previously disclosed, an exact comparison function may be a string distance function as previously described with a threshold set to only indicate a match when the compared strings are exactly matching. In decision block 424 the output of the exact comparison function is evaluated, for example, by comparing the output of the output the exact comparison function to what the strings matched. If the comparison yield true strings may be determined to match and method 235 may proceed to block 244. If the comparison yields false, method 235 may proceed to block 243.

After method 235 reaches block 243 or 244, method 235 may proceed to decision block 245. In decision block 245 it may be determined if there are additional strings to compare between the normalized data and dataset retrieved. If there are more strings to compare, method 236 may return to block 236 to select a new set of stings with matching field names that have not yet been compared. If there are no more strings to compare, method 235 may proceed to end block 246.

Once the patient identification data has been compared, the medial data from the electronic healthcare record may be added to the healthcare organization's database if the patient identification data meets or exceeds comparison requirements. Comparison requirements may vary based on an individual healthcare organization's preferences. An example comparison requirement is illustrated in Table 1. Comparison requirements in Table 1 require exact matches for date of birth, zip code, and phone number, and either exact matching or fuzzy matching with threshold of greater than 0.92 using a Jaro-Winkler string distance function on first name, last name, and address. In an alternate embodiment, comparison requirements may comprise fuzzy matching with threshold of greater than 0.92 on all fields except for date of birth. One of ordinary skill in the art will appreciate that any field may have any comparison requirement for a particular application.

TABLE 1

| Field | Requirement |
|---|---|
| First Name | Exact or Jaro Winkler Distance > 0.92 |
| Last Name | Exact or Jaro Winkler Distance > 0.92 |
| Date of Birth | Exact |
| Address | Exact or Jaro Winkler Distance > 0.92 |
| Zip | Exact |
| Phone | Exact |

If the identification data meets the selected comparison requirements, the medial data from the electronic healthcare record may be added to the healthcare organization's database and the medical data may be associated with the patient.

### EXAMPLES

The following examples are illustrative embodiments of particular features of the present disclosure. There may be many alternative methods or means to practice the teachings of present disclosure that fall within the scope of the present disclosure.

### Example 1

Example 1 will illustrate an embodiment of the records matching process of the present disclosure. A first patient record will be analyzed and compared against a database to determine if the patient is in the database. An illustrative electronic healthcare record for the first patient sourced from a clinical document architecture is displayed in Table 2. Although only certain fields, specifically a small subset of potential patient identifier fields, are explicitly shown in Table 2, one of ordinary skill in the art will understand that there may be any number of patient identifier fields and corresponding data in the electronic healthcare record. Additionally, for sake of brevity, the present illustrative example does not contain any medical data fields that may accompany actual electronic healthcare records.

TABLE 2

| | Clinical Document |
|---|---|
| Field | Architecture Data |
| First Name | John J. |
| Last Name | Smith |
| Date of Birth | 07261942 |
| Address - Street | 132 Elm Lane |
| ZIP Code | 65619-1234 |
| Phone | 8645555555 |

As previously disclosed, data may be normalized before comparison. Table 3 contains data from Table 2 after normalization. The data in Table 2 was normalized by the methods previously disclosed including by transforming all characters to lowercase, removing white space characters, applying a postal address verification standard to the address, and appending a 2 to the phone number.

TABLE 3

| | Clinical Document | Normalized Clinical |
|---|---|---|
| Field | Architecture Data | Document Architecture Data |
| First Name | John J. | johnj |
| Last Name | Smith | smith |
| Date of Birth | 07261942 | 19420726 |
| Address - Street | 132 Elm Lane | 132elmln |
| ZIP Code | 65619-1234 | 65619 |
| Phone | 8645555555 | 18645555555 |

As previously disclosed, electronic healthcare records sourced from a clinical document architecture must be matched to a patient before the electronic healthcare record can be added to a healthcare organization's database for the particular patient. Table 4 shows an illustrative electronic healthcare record for a patient retrieved from a database of a healthcare organization. As with Table 2, only a partial list of potential patient identifier fields are listed. Table 2 comprises a first column with original database data which is the data retrieved from the healthcare organization's database before normalization operations are performed on the data. Table 2 further comprises a second column with a normalized version of the data from the first column. The data has been normalized by the methods previously disclosed.

TABLE 4

| Field | Database Data | Normalized Database Data |
|---|---|---|
| First Name | John | john |
| Last Name | Smith | smith |
| Date of Birth | 07261942 | 19420726 |
| Address - Street | 132 Elm | 132elm |
| ZIP Code | 65619-1234 | 65619 |
| Phone | 8645555555 | 18645555555 |

Table 5 shows a comparison of the normalized clinical document architecture data and normalized database data. As previously disclosed, comparisons may be fuzzy or exact depending on the selected field. An exact compare was selected for last name, date of birth, zip code, and phone number and a fuzzy compare was selected for first name and address. The exact comparison function selected was a compare sting function. The compare string function was applied to each selected exact compare field to determine if the data in the corresponding field of the normalized clinical document architecture data matches that of the normalized database data. A fuzzy match Jaro-Winkler distance string metric was applied to each selected fuzzy comparison field to determine a similarity score. A threshold of 0.92 or greater was selected as the minimum similarity score to determine if the strings matched. With the aforementioned method each field was determined to match either by an exact match using a compare function or by exceeding the minimum similarity threshold. It was determined that the electronic healthcare record sourced from the clinical document architecture and the electronic healthcare record database data were a match as each corresponding field was determined to be an exact match by sting comparison or a match by exceeding the threshold similarity score.

TABLE 6

| | Normalized | | | |
|---|---|---|---|---|
| | Clinical | | | |
| | Document | Normalized | | |
| | Architecture | Database | | |
| Field | Data | Data | Comparison | Result |
| First Name | johnj | john | Jaro- | 0.97 |
| | | | Winkler > 0.92 | Match |
| Last Name | smith | smith | Exact | Match |
| Date of | 19420726 | 19420726 | Exact | Match |
| Birth | | | | |
| Address - | 132elmln | 132elm | Jaro- | 0.98 |
| Street | | | Winkler > 0.92 | Match |
| ZIP Code | 65619 | 65619 | Exact | Match |
| Phone | 18645555555 | 18645555555 | Exact | Match |

Example 2 will illustrate an electronic healthcare record comparison that does not match as in Example 2. An illustrative electronic healthcare record for a second patient sourced from a clinical document architecture is displayed in Table 6. As with Table 2, only a small subset of potential patient identifier fields are explicitly listed. One of ordinary skill in the art will understand that there may be any number of patient identifier fields and corresponding data in the electronic healthcare record. Table 6 comprises a first column with the second patient data sourced from the clinical document architecture and a second column with the equivalent normalized data. The data was normalized by the methods enumerated in Example 2.

TABLE 6

| | | Normalized Clinical |
|---|---|---|
| | Clinical Document | Document Architecture |
| Field | Architecture Data | Data |
| First Name | James | james |
| Last Name | Smith | smith |
| Date of Birth | 10051995 | 19951005 |
| Address - Street | 132 Elm Lane | 132elmln |
| ZIP Code | 65619-1234 | 65619 |
| Phone | 8645555555 | 18645555555 |

As in Example 1, electronic healthcare records sourced from a clinical document architecture are compared to patient data from a healthcare organization's database. Table 7 compares normalized clinical document architecture from Table 6 for patient 2 to normalized database data from Table 4 for patient 1.

TABLE 7

| | Normalized Clinical | Normalized |
|---|---|---|
| | Document Architecture | Database Data - |
| Field | Data - Patient 2 | Patient 1 |
| First Name | james | johnj |
| Last Name | smith | smith |
| Date of Birth | 19951005 | 19420726 |
| Address - Street | 132elmln | 132elmln |
| ZIP Code | 65619 | 65619 |
| Phone | 18645555555 | 18645555555 |

Table 8 shows a comparison of the normalized clinical document architecture data and normalized database data for patient 1 and patient 2 from Table 7. The same fuzzy comparison and exact comparison fields were selected as in Example 1. The compare string function was applied to each selected exact compare field to determine if the data in the corresponding field of the normalized clinical document architecture data matches that of the normalized database data. A fuzzy match Jaro-Winkler distance string metric was applied to each selected fuzzy comparison field to determine a similarity score. A threshold of 0.92 or greater was selected as the minimum similarity score to determine if the strings matched. With the aforementioned method each field was determined to match either by an exact match using a compare function or by exceeding the minimum similarity threshold. It was determined that the electronic healthcare record sourced from the clinical document architecture for patient 2 and the electronic healthcare record database data for patient 1 were not a match as each field did not match by exact or fuzzy matching.

TABLE 8

| | Normalized | | | |
|---|---|---|---|---|
| | Clinical | Normalized | | |
| | Document | Database | | |
| | Architecture | Data - | | |
| Field | Data - Patient 2 | Patient 1 | Comparison | Result |
| First Name | james | johnj | Jaro- | 0.47, |
| | | | Winkler > 0.92 | Fail |
| Last Name | smith | smith | Exact | Match |
| Date of | 19951005 | 19420726 | Exact | Fail |
| Birth | | | | |
| Address - | 132elmln | 132elmln | Jaro- | 0.98 |
| Street | | | Winkler > 0.92 | Match |
| ZIP Code | 65619 | 65619 | Exact | Match |
| Phone | 18645555555 | 18645555555 | Exact | Match |

In embodiments, the methods disclosed herein may be referred to as an interoperable record matching process. The interoperable record matching process may be performed by a computer system with software running on said computer system, wherein the software is configured to perform the methods disclosed herein. The computer system may comprise any computer systems such as local computers, computers connected over the internet, cloud based computers, and any other computing devices configured to be part of the system described herein.

In embodiments, the interoperable records matching process may match an electronic healthcare record to a patient. Once the record is matches to a patient, the electronic healthcare record may be validated by a document validator to ensure the electronic healthcare record complies with a particular clinical document architecture. An example of a document validator may be the Model Driven Health Tools project. The document validator may format the electronic healthcare record to match requirements of a particular clinical document architecture. Once an electronic healthcare record is validated, it may be added to a healthcare organization's database.

As previously mentioned, some medical reimbursement programs, such as MIPS, may have document format requirements and reporting requirements. Access to a particular patient medical record in a healthcare organization's database may be recorded as action events to the medical record. Actions may include viewing, downloading, modifying, or transmitting. The action events may be recorded and then later queried by the healthcare provider or organization to submit to the centers for Medicare and Medicaid Services for attestation or to any other entity requiring validation/attestation of the records. The action events may be queried at the individual patient level, healthcare provider level, healthcare location level, and healthcare organization level.

Although the present invention and its advantages have been described in detail, it should be understood that various changes, substitutions and alterations may be made herein without departing from the spirit and scope of the invention. For the sake of brevity, only certain ranges are explicitly disclosed herein. However, ranges from any lower limit may be combined with any upper limit to recite a range not explicitly recited, as well as, ranges from any lower limit may be combined with any other lower limit to recite a range not explicitly recited, in the same way, ranges from any upper limit may be combined with any other upper limit to recite a range not explicitly recited. Additionally, whenever a numerical range with a lower limit and an upper limit is disclosed, any number and any included range falling within the range are specifically disclosed. In particular, every range of values (of the form, “from about a to about b,” or, equivalently, “from approximately a to b,” or, equivalently, “from approximately a-b”) disclosed herein is to be understood to set forth every number and range encompassed within the broader range of values even if not explicitly recited. Thus, every point or individual value may serve as its own lower or upper limit combined with any other point or individual value or any other lower or upper limit, to recite a range not explicitly recited.

1. A method comprising:
receiving an electronic healthcare record comprising patient identification data and medical data;
parsing the electronic healthcare record and extracting the patient identification data to produce extracted patient identification data;
normalizing the extracted patient identification data to produce normalized patient identification data;
retrieving a patient data file from a database wherein the patient data file comprises database patient identification data;
comparing the normalized patient identification data to the database patient identification data using a string distance function, wherein the string distance function computes a string distance;
comparing the distance metric to a threshold; and
adding the medical data to the database and associating the medical data with the database patient identification data if the string distance meets or exceeds the threshold.

2. The method of claim 1 wherein the step of receiving the electronic healthcare record comprises receiving from a direct trusted protocol network, a blue button+network, an application program interface, an e-mail or e-mail attachment, cloud storage, electronic heath record software, or combinations thereof.

3. The method of claim 1 wherein the step of receiving is performed over an internet connection.

4. The method of claim 1 wherein the healthcare record comprises a clinical document architecture format.

5. The method of claim 1 wherein the patient identification data comprises a first name, a last name, a birth date, a street address, a zip code, a telephone number, an e-mail address, a unique organization identifier, a healthcare provider national provider identifier, or combinations thereof.

6. The method of claim 1 wherein the step of normalizing comprises removing all characters except for letters and numbers, transforming uppercase letters into lowercase letters, transforming lowercase letters into uppercase letters, removing white space characters, removing glyphs from letters to generate basic glyphs, applying a postal address verification standard, or combinations thereof.

7. The method of claim 1 wherein the database patient identification data comprises a first name, a last name, a birth date, a street address, a zip code, a telephone number, an e-mail address, a unique organization identifier, a healthcare provider national provider identifier, or combinations thereof

8. The method of claim 1 wherein database patient identification data comprises normalized data.

9. The method of claim 1 further comprising normalizing the database patient identification data.

10. The method of claim 1 wherein the string distance function is Levenshtein distance, Jaro-Winkler distance, Damerau-Levenshtein distance, or Hamming distance.

11. The method of claim 1 wherein the step of comparing the normalized patient identification data to the database patient identification data comprises:
selecting a first string with a first field type from the normalized patient identification data;
selecting a second string from the database patient identification data, wherein the second string comprises a second field type that is equal to the first field type; and
comparing the first string and the second string using the string distance function.

12. The method of claim 1 wherein the string distance function is Jaro-Winkler distance and the threshold is 0.9.

13. The method of claim 1 further comprising validating the medical data using a document validator before the step of adding the medical data to the database.

14. The method of claim 13 wherein the document validator formats the medical data to a clinical document architecture.

15. A system comprising:
a source of an electronic healthcare record, the electronic healthcare record comprising patient identification data and medical data;
a database comprising a patient data file, the a patient data file comprising database patient identification data;
a computer system in communication with the source and the database, the computer system configured to:
receive the electronic healthcare record and the patient data file;
extract the patient identification data from the electronic healthcare record to produce extracted patient identification data;
normalize the extracted patient identification data to produce normalized patient identification data;
compute a string distance between the normalized patient identification data and the extracted patient identification data; and
add the medical data to the database.

16. The system of claim 15 wherein the source of the electronic healthcare record comprises a direct trusted protocol network, a blue button+network, an application program interface, an e-mail or e-mail attachment, a cloud storage platform, an electronic heath record software, or combinations thereof

17. The system of claim 15 wherein computer system is configured to normnalize the extracted patient identification data by removing all characters except for letters and numbers, transforming uppercase letters into lowercase letters, transforming lowercase letters into uppercase letters, removing white space characters, removing glyphs from letters to generate basic glyphs, applying a postal address verification standard, or combinations thereof.

18. The system of claim 15 wherein the computer system is configured to compute the string distance using a Levenshtein distance function, Jaro-Winkler distance function, Damerau-Levenshtein distance function, or Hamming distance function.

19. The system of claim 15 wherein the computer system is further configured to validate the medical data.

20. The system of claim 19 wherein the validation comprises formatting the medical data to a clinical document architecture format.
